(19) United States
(12) Patent Application Publication
(10) Pub. No.: US 2010/0019176 A1
(43) Pub. Date: Jan. 28, 2010

(54) IMAGING APPARATUS

(76) Inventor: Koichi Tanabe, Kyoto (JP)

Correspondence Address: Cheng Law Group, PLLC, 1100 17th Street, N.W., Suite 503, Washington, DC, 20036, US

(21) Appl. No.: 12/518,559

(22) PCT Filed: Dec. 12, 2006
(86) PCT No.: PCT/JP2006/324757

§ 371 Date: Jun. 10, 2009

(51) Int. Cl. G01N 23/04

(52) U.S. Cl. 250/591

(57) ABSTRACT

With the imaging apparatus of this invention, an image is divided equally into four areas, and a setting is made for reading of carriers before irradiation to be carried out separately according to images of the divided areas. By dividing the reading of carriers before irradiation in this way, when compared with the reading of carriers for an entire area of an image (i.e. a frame) in the prior art, each storage and reading time can be shortened to one of the number of divisions. A time serving as a starting point of an irradiation wait time occurs before the reading of carriers before irradiation. Consequently, even if the time serving as the starting point of the irradiation wait time varies, the variation takes place only during each storage and reading set short. Thus, the variation of the irradiation wait time is made less than in the prior art, thereby improving response.

### TECHNICAL FIELD

This invention relates to an imaging apparatus for use in the medical field, industrial field, nuclear field and so on.

### BACKGROUND ART

An imaging apparatus that obtains images based on detected light or radiation has a light or radiation detector for detecting light or radiation. An X-ray detector will be described by way of example. An X-ray detector has an X-ray converting layer (semiconductor layer) of the X-ray sensitive type. The X-ray conversion layer converts incident X rays into carriers (charge information). The detector detects the X rays by reading the converted carriers. Amorphous selenium (a-Se) film, for example, is used as the X-ray conversion layer (see Nonpatent Document 1, for example).

In a radiographic operation carried out by irradiating a subject with X rays, radiographic images transmitted through the subject are projected onto the amorphous selenium film, thereby generating carriers proportional to the densities of the images in the film. Subsequently, the carriers generated in the film are collected by carrier collecting electrodes in a two-dimensional arrangement. After the collection is continued for a predetermined time (called “storage time”), the carriers are read outside via thin-film transistors.

Such an X-ray detector has peripheral circuits such as a gate driver circuit for turning the thin-film transistor switches on and off, and an amplifier array circuit for reading the carriers. The driver circuit applies a driving signal to the X-ray detector to drive the X-ray detector. The amplifier array circuit receives the carriers read based on a read signal relating to reading of the carriers. The X-ray detector and these circuits constitute an image sensor.

For reading the carriers, there are a method of reading one line at a time through data lines, and a method of reading a plurality of lines through the data lines. In the case of the former method of reading one line at a time, the thin-film transistor switches are turned on and driven one at a time (or turned off one at a time), and carriers once stored in capacitors connected to the switches driven are read one line at a time through the data lines connected to the switches. In the case of the latter method of reading a plurality of lines, on the other hand, a plurality of the thin-film transistor switches are turned on and driven simultaneously (or two or more are turned off simultaneously), and carriers once stored in the capacitors connected to the switches driven simultaneously are read en bloc through the data lines connected to these switches.

Since carriers are stored in the capacitors due to leak currents (also called “dark currents”) of the amorphous selenium film even when X rays are not emitted, it is necessary to read the carriers of X-ray non-irradiation times (also called “dark image information”) by driving the thin-film transistors periodically. A correction is carried out based on this dark image information read (also called “dark correction” or “offset correction”). Also for reading the dark image information which consists of the carriers of non-irradiation times, either the former method of reading one line at a time or the latter method of reading a plurality of lines can be used.

[Nonpatent Document 1]

W. Zhao, et al., “A flat panel detector for digital radiology using active matrix readout of amorphous selenium,” Proc. SPIE Vol. 2708, pp. 523-531, 1996.

### DISCLOSURE OF THE INVENTION

[Problem to be Solved by the Invention]

The former method of reading one line at a time has a problem that response is slow since the carriers are successively read one line at a time. The latter method of reading by a plurality of lines, although it can read at higher speed than the former method of reading one line at a time, has a problem that dark image information immediately before irradiation cannot be acquired, and a problem that image artifacts are produced by changing the number of the lines driven at the same time. In any case, apart from the two techniques, it is desired to improve response by using a different technique.

A cause of the slow response is variations in irradiation wait time. The variations in irradiation wait time will be described with reference to FIG. 17. FIG. 17 is a timing chart of frame rates and signals relating thereto in the prior art.

Frame rates T are cycles relating to a series of operations for storage and reading of carriers. During these frame rates T, carriers for frames are read (see F1-F3 for image reading in FIG. 17). Times other than the image reading are the times when X-ray irradiation is enabled (X-ray irradiation enable times in FIG. 17). Specifically, operation is started in the order of F1-F3 in FIG. 17. As shown in FIG. 17, assuming that the time of image reading for each frame rate T is a “read period”, the time other than the read period in each frame rate T is a time when X-ray irradiation is enabled. This time when X-ray irradiation is enabled is called “X-ray irradiation enable time”. Carrier storage for each frame is carried out in each frame rate T, including the read period and X-ray irradiation enable time. Assuming the read period to be t_{READ} and the X-ray irradiation enable time to be t_{IRRA}, T=t_{READ}+t_{IRRA} is established as clearly seen from the reason set out above. In FIG. 17, the read period t_{READ} is set to 240 ms, and the frame rate T to 267 ms.

In actually irradiating X rays, X-ray pulses are emitted during the X-ray irradiation enable times. Also at non-irradiation times before start of X-ray irradiation, reading for the frames usually is carried out as shown in FIG. 17, in order to release leak currents occurring at the non-irradiation times, and X-ray irradiation enable times t_{IRRA} are set as are irradiation times.

A hand switch is provided to make a shift to preparation for X-ray irradiation, at the non-irradiation times before start of X-ray irradiation. A shift is made to preparation for X-ray irradiation when the hand switch is depressed at a time A in FIG. 17. In the case of FIG. 17, an X-ray irradiation enable signal is continuously outputted without synchronizing with a first frame synchronization signal outputted after the shift to preparation for X-ray irradiation, and is stopped synchronously with a frame synchronization signal outputted next. As a result, only for a period from time A to stopping of the X-ray irradiation enable signal, the X-ray irradiation enable time is longer than the usual X-ray irradiation enable time t_{IRRA}. X-ray pulses are emitted during this extended X-ray irradiation enable time.

Specifically, X-ray pulses are emitted at time B in FIG. 17, which is synchronized with the first frame synchronization signal outputted after the shift to preparation for X-ray irradiation, or synchronized with a predetermined time from this frame synchronization signal (the predetermined time being less than frame rate T). The emission of X-ray pulses is stopped by the time the X-ray irradiation enable signal is stopped synchronously with the frame synchronization signal outputted next. Carriers are read for a frame (see F3 in FIG. 17) immediately after the X-ray pulses are emitted, and imaging is carried out using the carriers read. The time from time A for depressing the hand switch to time B for outputting X-ray pulses is called “irradiation wait time”. The irradiation wait time is referenced t_{WAIT}.

In FIG. 17, the X-ray irradiation enable signal is continuously outputted without synchronizing with the first frame synchronization signal outputted after the shift to preparation for X-ray irradiation, and is stopped synchronously with the frame synchronization signal outputted next. This is not limitative. The X-ray irradiation enable signal may be continuously outputted without synchronizing with the frame synchronization signal outputted next, and may be stopped synchronously with a frame synchronization signal outputted after the next, thereby to extend the X-ray irradiation enable time and to set the emission of X-ray pulses to be long. Thus, by increasing the periodic number of the frame synchronization signal with which stopping of the X-ray irradiation signal is synchronized, the X-ray irradiation enable time can be extended further to set the emission of X-ray pulses to be still longer.

When carrying out the dark correction noted hereinbefore, as shown in FIG. 18, carriers for the frame (see the hatched frame in FIG. 18) read with the same timing as in FIG. 17 and without outputting X-ray pulses are read as carriers of an X-ray non-irradiation time. The dark correction is carried out using the read carriers as dark image information. FIG. 18 is a timing chart of signals relating to reading of dark image information in the prior art.

Specifically, as shown in FIG. 17, t refers to a storage time from start of the frame (see F2 in FIG. 17) before the frame to be imaged to start of the frame to be imaged (that is, the frame immediately after X-ray pulses are outputted: see F3 in FIG. 17). The characteristic of dark image information changes depending on the length of this storage time t. Thus, as shown in FIG. 18, a storage time from start of the frame before the frame for reading dark image information to start of the frame for reading dark image information (see the hatched frame in FIG. 18) is set to the same storage time t to be the same storage time t in FIG. 17. Although X-ray pulses are outputted between these frames in ordinary imaging, X-ray pulses are not outputted when reading dark image information as shown in FIG. 18.

Returning to the description of the irradiation wait time, such hand switch is depressed manually, and therefore the shift to preparation of X-ray irradiation does not synchronize with a frame synchronization signal. Consequently, as shown in FIG. 19(a), for example, when the hand switch is depressed halfway through the reading for frame F2, the X-ray irradiation enable signal is continuously outputted without synchronizing with the first frame synchronization signal outputted immediately after frame F2, and is stopped synchronously with the frame synchronization signal outputted next. X-ray pulses are outputted at time B synchronized with this frame synchronization signal or synchronized with a predetermined time from this frame synchronization signal.

On the other hand, as shown in FIG. 19(b), when the hand switch is depressed immediately after start of reading for the next frame F3, the X-ray irradiation enable signal is continuously outputted without synchronizing with the first frame synchronization signal outputted immediately after frame F3, and is stopped synchronously with the frame synchronization signal outputted next. X-ray pulses are outputted at time B synchronized with this frame synchronization signal or synchronized with a predetermined time from this frame synchronization signal.

This irradiation wait time t_{WAIT} is variable up to a maximum corresponding to the frame rate T as shown in FIG. 19. Therefore, where the subject is a patient and X-ray irradiation is timed with the patient's respiration, there occurs a problem that it is difficult to carry out X-ray irradiation matched with respiratory timing.

This invention has been made having regard to the state of the art noted above, and its object is to provide an imaging apparatus that can improve response.

[Means for Solving the Problem]

To fulfill the above object, this invention provides the following construction.

An imaging apparatus of this invention is an imaging apparatus for obtaining images by carrying out imaging based on light or radiation, comprising a conversion layer for converting light or radiation information to charge information in response to incident light or radiation, and a storage and reading circuit for storing and reading the charge information converted by the conversion layer, the apparatus being constructed to obtain the images based on the charge information read by the storage and reading circuit, the apparatus further comprising a first storage and readout setting device for dividing an image into a plurality of predetermined areas, and setting storage and reading of the charge information before irradiation of the light or radiation according images of the divided areas.

According to the imaging apparatus of this invention, the first storage and readout setting device divides an image into a plurality of predetermined areas, and sets for storage and reading of the charge information before the irradiation of light or radiation to be carried out separately according images of the divided areas. By dividing the storage and reading of the charge information before the irradiation in this way, when compared with the storage and reading of charge information for an entire area of an image in the prior art, an average time for the storage and reading can be shortened to one of the number of divisions. A time serving as a starting point of an irradiation wait time occurs before the storage and reading of the charge information before the irradiation. Consequently, even if the time serving as the starting point of the irradiation wait time varies, the variation takes place only during each storage and reading time set short. Thus, the variation of the irradiation wait time is made less than in the prior art, thereby improving response.

One example of the above invention (the former) provides a storage and readout stopping device for stopping the storage and reading of the charge information before the irradiation, even if the storage and reading of the charge information before the irradiation is only halfway through an image, according to the image of a divided area corresponding to the halfway area, and an irradiation control device for controlling to carry out the irradiation after the storage and reading of the charge information before the irradiation are stopped by the storage and readout stopping device.

According to this example, even if the storage and reading of the charge information before the irradiation is only halfway through an image, the storage and readout stopping device can stop the storage and reading of the charge information before the irradiation according to the image of a divided area corresponding to the halfway area. And after the storage and reading of the charge information before the irradiation are stopped by the storage and readout stopping device, the irradiation control device controls to carry out the irradiation. Thus, the irradiation of light or radiation can be carried out even when the storage and reading of the charge information before the irradiation is only halfway through an image.

Another example of the above invention (the latter) provides a second readout setting device for reading the charge information before the irradiation periodically, and interposing, in an arbitrary cycle, a non-reading operation between the reading in that cycle and reading in a following cycle. With such second readout setting device, application can be made to an imaging apparatus that carries out control synchronously with the cycles. The non-reading operation may be set to the storage, or the reading and the non-reading operation may be set to the storage.

This other example (the latter) may provide an irradiation control device as in the former example. That is, it provides a readout stopping device for stopping the reading of the charge information before the irradiation, even if the reading of the charge information before the irradiation is only halfway through an image, according to the image of a divided area corresponding to the halfway area, and synchronously with a cycle corresponding to halfway timing, and an irradiation control device for controlling to carry out the irradiation after the reading of the charge information before the irradiation is stopped by the readout stopping device, and at a time of non-reading operation.

In this other example (the latter), where the irradiation control device is provided, even if the reading of the charge information before the irradiation is only halfway through an image, the readout stopping device can stop the reading of the charge information before the irradiation according to the image of a divided area corresponding to the halfway area. And after the reading of the charge information before the irradiation is stopped by the readout stopping device, and at a time of non-reading operation, the irradiation control device controls to carry out the irradiation. Thus, the irradiation of light or radiation can be carried out even when the reading of the charge information before the irradiation is only halfway through an image.

In the latter example, where the irradiation control device is provided, it is preferred that the reading of the charge information before the irradiation is carried out periodically in order of divided adjoining areas, and when a last area is finished, a return is made to a first area for repetition. In this way, the reading of the charge information before the irradiation can be repeated.

In the latter example, where the irradiation control device is provided, and in one example of repeating the reading of the charge information before the irradiation, the reading of the charge information at an irradiation time is started from a next area adjacent the area where the reading of the charge information is stopped, the reading of the charge information at the irradiation time is carried out periodically in the order of the divided adjoining areas from the starting area, and when the last area is finished, a return is made to the first area for repetition.

According to this example, the reading of the charge information at an irradiation time can be started from a next area adjacent the area where the reading of the charge information is stopped. Even if the next area which is an area for starting the reading of the charge information at the irradiation time is not the first area, a return is made to the first area for repetition when the last area is finished. Thus, the reading of the charge area at the irradiation time can be carried out for all the areas.

In the latter example, where the irradiation control device is provided, another example of repeating the reading of the charge information before the irradiation provides an area changing device capable of changing areas where the reading of carriers at the irradiation time is started, wherein the reading of the charge information at the irradiation time is carried out periodically in the order of the divided adjoining areas from the starting area, and when the last area is finished, a return is made to the first area for repetition.

According to this other example, with the area changing device changing areas for starting the reading of carriers at the irradiation time, the reading of the charge information at the irradiation time can be started from any arbitrary area. Even if the arbitrary area which is an area for starting the reading of the charge information at the irradiation time is not the first area, a return is made to the first area for repetition when the last area is finished. Thus, the reading of the charge area at the irradiation time can be carried out for all the areas.

One example of area changes by the area changing device is to start the reading of the charge information from the first area. A luminance difference occurring at a boundary between divided images when reading the charge information from intermediate areas can be solved by starting the reading of the charge information at the irradiation time from the first area.

In the latter example, where the irradiation control device is provided, and in a further example of repeating the reading of the charge information before the irradiation, the reading of the charge information is carried out continuously according to all areas of the image.

According to this further example, the reading of the charge information at the irradiation time is carried out continuously according to all the areas of the image, whereby the reading is carried out faster than the reading of the charge information before the irradiation. As long as the storage and reading of the charge information before the irradiation are carried out separately in this invention, the question of response which is the problem addressed by this invention can be solved. Thus, the reading of the charge information at the irradiation time may be carried out continuously according to all the areas of an image.

One example of these inventions provides a correcting device for correcting charge information read at an irradiation time based on charge information read at a non-irradiation time of the light or radiation. This invention is applicable where the charge information is corrected based on the charge information read at the non-irradiation time (dark image information). The non-irradiation time here may be before the irradiation noted above, or may be after the irradiation. That is, the charge information read at the non-irradiation time for use in the correction may be charge information read before the irradiation, or may be charge information read after the irradiation.

The charge information read at the non-irradiation time for use in the correction may consist of more than one piece. In the former and latter examples in this invention, the start of irradiation is determined by the time of stopping the storage and reading or stopping the reading, and the time of starting the irradiation is unknown. Therefore, considering that the time of starting the irradiation is unknown, plural pieces of charge information matched with times of starting the irradiation are provided, thereby carrying out the above-noted correction with increased accuracy.

### EFFECTS OF THE INVENTION

According to the imaging apparatus of this invention, a time serving as a starting point of an irradiation wait time occurs before the storage and reading of charge information before irradiation. Consequently, even if the time serving as the starting point of the irradiation wait time varies, the variation takes place only during each storage and reading time set short. Thus, the variation of the irradiation wait time is made less than in the prior art, thereby improving response.

### BRIEF DESCRIPTION OF THE DRAWINGS

FIG. 1 is a block diagram of an X-ray apparatus according to each embodiment;

FIG. 2 shows an equivalent circuit, seen in side view, of a flat panel X-ray detector used in the X-ray apparatus;

FIG. 3 shows an equivalent circuit, seen in plan view, of the flat panel X-ray detector;

FIG. 4 is a timing chart of frame rates and signals relating thereto according to Embodiment 1;

FIG. 5 is a schematic view of an image divided into four;

FIGS. 6(a) and (b) are timing charts of signals before and after a hand switch is depressed in the timing chart of FIG. 4;

FIG. 7 is an explanatory view of dark image information according to Embodiment 1;

FIG. 8 is a timing chart of frame rates and signals relating thereto according to Embodiment 2;

FIG. 9 is an explanatory view of dark image information according to Embodiment 2;

FIG. 10 is a timing chart of frame rates and signals relating thereto according to Embodiment 3;

FIG. 11 is an explanatory view of dark image information according to Embodiment 3;

FIG. 12 is a timing chart of frame rates and signals relating thereto according to Embodiment 4;

FIG. 13 is an explanatory view of dark image information according to Embodiment 4;

FIG. 14 is a timing chart of frame rates and signals relating thereto according to a modification.

FIG. 15 is a schematic view of an image dividing mode according to a further modification;

FIG. 16 is a timing chart of frame rates and signals relating thereto according to the further modification;

FIG. 17 is a timing chart of frame rates and signals relating thereto in the prior art;

FIG. 18 is a timing chart of signals relating to reading of dark image information in the prior art; and

FIGS. 19(a) and (b) are timing charts of signals before and after a hand switch is depressed in the timing chart of FIG. 17.

### DESCRIPTION OF REFERENCES

7 . . . X-ray tube controller

8 . . . analog-to-digital converter

9 . . . image processor

10 . . . controller

31 . . . semiconductor thick film

37 . . . amplifier array circuit

Ca . . . capacitors

D1, D2, D3, D4 . . . areas

t_{READ} . . . read period

T . . . frame rates

S . . . image sensor

### Embodiment 1

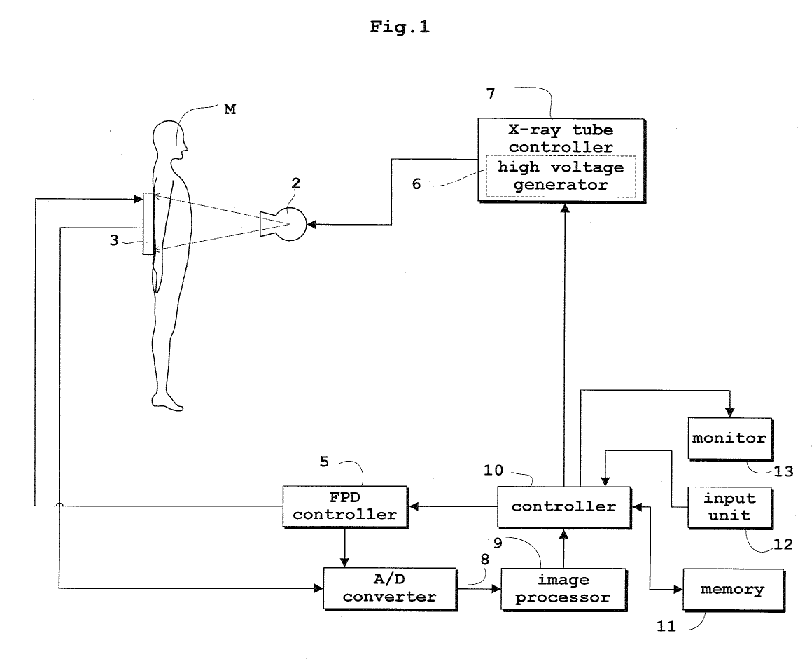

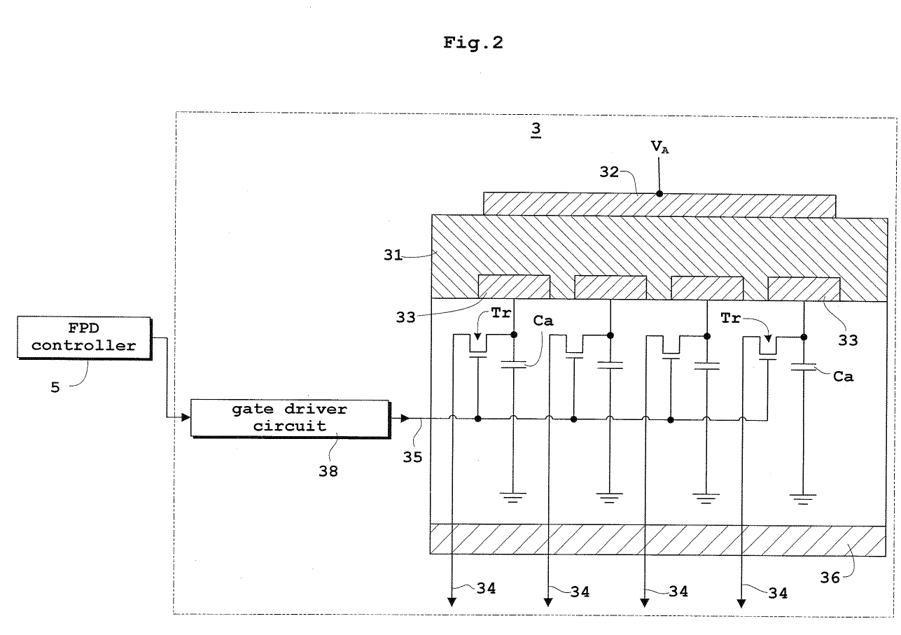

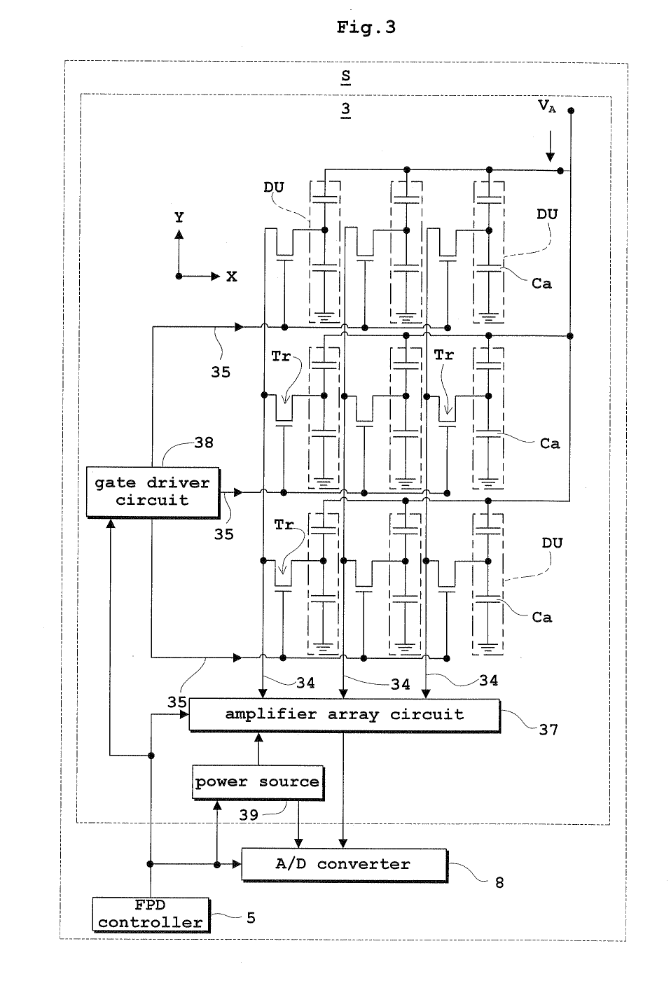

Embodiment 1 of this invention will be described hereinafter with reference to the drawings. FIG. 1 is a block diagram of an X-ray apparatus according to each embodiment. FIG. 2 is an equivalent circuit, seen in side view, of a flat panel X-ray detector used in the X-ray apparatus. FIG. 3 is an equivalent circuit, seen in plan view, of the flat panel X-ray detector. Embodiment 1, including Embodiments 2-4 to follow, will be described, taking the flat panel X-ray detector (hereinafter called “FPD” as appropriate) as an example of light or radiation detector, and the X-ray apparatus as an example of imaging apparatus. The X-ray apparatus and FPD in each embodiment are constructed as shown in FIGS. 1-3.

As shown in FIG. 1, the X-ray apparatus according to Embodiment 1, including Embodiments 2-4 to follow, has an X-ray tube 2 for emitting X rays toward a patient M, and an FPD 3 for detecting X rays transmitted through the patient M.

The X-ray apparatus further includes an FPD controller 5 for controlling scanning action of the FPD 3, an X-ray tube controller 7 having a high voltage generator 6 for generating a tube voltage and tube current for the X-ray tube 2, an analog-to-digital converter 8 for fetching X-ray detection signals which are charge signals from the FPD 3 and digitizing the signals, an image processor 9 for performing various processes based on the X-ray detection signals outputted from the analog-to-digital converter 8, a controller for performing an overall control of these components, a memory 11 for storing processed images, an input unit 12 for the operator to input settings, and a monitor 13 for displaying the processed images and the like.

The FPD controller 5 controls scanning action by moving the FPD 3 horizontally or revolving the FPD 3 about the body axis of patient M. The high voltage generator 6 generates the tube voltage and tube current for the X-ray tube 2 to emit X rays. The X-ray tube controller 7 controls scanning action by moving the X-ray tube 2 horizontally or revolving the X-ray tube 2 about the body axis of patient M, and controls setting of a coverage of a collimator (not shown) disposed adjacent the X-ray tube 2. In time of scanning action of the X-ray tube 2 and FPD 3, the X-ray tube 2 and FPD 3 are moved while maintaining a mutually opposed relationship, so that the FPD 3 may detect X rays emitted from the X-ray tube 2.

The controller 10 has a central processing unit (CPU) and other elements. The memory 11 has storage media, typically a ROM (Read-Only Memory) and a RAM (Random Access Memory). The input unit 12 has a pointing device, typically a mouse, keyboard, joy stick, trackball and/or touch panel. The X-ray apparatus creates images of the patient M, with the FPD 3 detecting X rays transmitted through the patient M, and the image processor 9 performing an image processing based on the X rays detected.

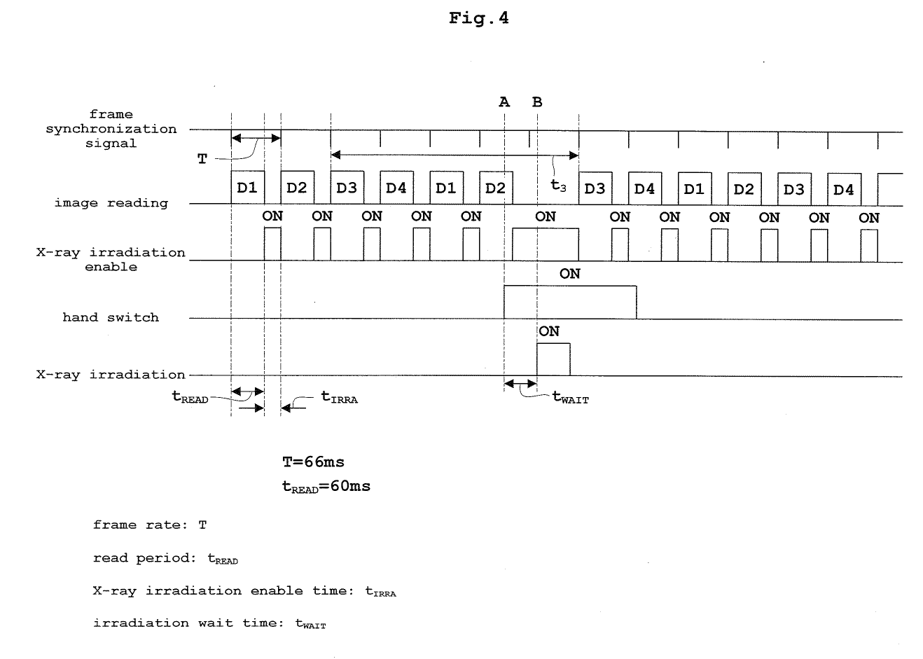

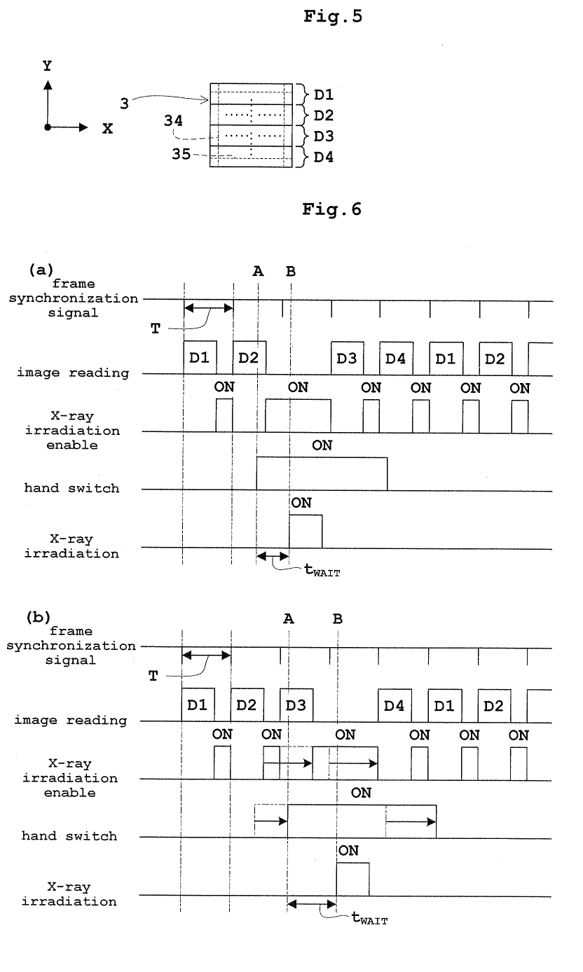

In Embodiment 1, the controller 10 has also a function, described hereinafter, to divide an image equally into four areas D1-D4 (see FIG. 5), and set for reading of carriers before irradiation separately according to images of the four divided areas D1-D4. Further, the controller 10 has also (1) a function to read carriers before irradiation periodically, and interpose, in an arbitrary cycle, a non-reading operation (X-ray irradiation enable in FIG. 4) between reading in that cycle and reading in the following cycle, and (2) a function to stop the reading of carriers before the irradiation, even if the reading of carriers before the irradiation is only halfway through an image, according to the image of a divided area (D2 in FIG. 4) corresponding to the halfway area, and synchronously with the cycle corresponding to the halfway timing. The controller 10 corresponds to the first storage and readout setting device, the second readout setting device and the readout stopping device in this invention.

In Embodiment 1, the X-ray tube controller 7 has a function to control the X-ray tube 2 to emit X rays after the reading of carriers before the irradiation is stopped halfway through an image and at the time of non-reading operation (X-ray irradiation enable). The X rays emitted from the X-ray tube 2 at this time are X-ray pulses. The X-ray tube controller 7 corresponds to the irradiation control device in this invention.

Regarding the memory 11, the RAM is used for writing of X-ray detection signals, processed images and so on, and the ROM is used for reading only of a program of control sequence when, for example, causing the controller 10 to perform the control sequence by reading the program of control sequence. Embodiment 1, including also Embodiments 2-4 to follow, causes the memory 11 to store the program of control sequence which sets for reading of carriers before the irradiation separately according to images of the four divided areas D1-D4, and causes the controller 10 to perform the control sequence by reading the program.

In Embodiment 1, including also Embodiments 2-4 to follow, the input unit 12 has a hand switch (not shown), and has a function to make a shift to preparation for X-ray irradiation by depressing the hand switch, and start X-ray irradiation after lapse of a predetermined time. Specifically, as shown in FIG. 4, a shift is made to preparation for X-ray irradiation when the hand switch is depressed at time A, and an X-ray irradiation enable signal is continuously outputted without synchronizing with a first frame synchronization signal outputted, and is stopped synchronously with a frame synchronization signal outputted next. X-ray pulses are emitted while the X-ray irradiation enable signal is outputted.

As shown in FIG. 2, the FPD 3 includes a radiation sensitive thick semiconductor film 31 for generating carriers in response to incident radiation such as X rays, a voltage application electrode 32 formed on the surface of thick semiconductor film 31, carrier collecting electrodes 33 arranged on the back surface remote from the radiation incidence side of the thick semiconductor film 31, carrier storing capacitors Ca for storing the carriers collected by the carrier collecting electrodes 33, and thin-film transistors (TFT) Tr acting as charge fetching switching elements, normally turned off (cutoff), for fetching the carriers (charges) from the capacitors Ca. In Embodiment 1, including also Embodiments 2-4 to follow, the thick semiconductor film 31 is formed of a radiation sensitive material which generates carriers in response to incident radiation, such as amorphous selenium, but may be formed of a light sensitive material for generating carriers in response to incident light. The thick semiconductor film 31 corresponds to the conversion layer in this invention.

In addition, Embodiment 1, including also Embodiments 2-4 to follow, provides data lines 34 connected to the sources of the thin-film transistors Tr, and gate lines 35 connected to the gates of the thin-film transistors Tr. The voltage application electrode 32, thick semiconductor film 31, carrier collecting electrodes 33, capacitors Ca, thin-film transistors Tr, data lines 34 and gate lines 35 are laminated on an insulating substrate 36.

As shown in FIGS. 2 and 3, the capacitors Ca and thin-film transistors Tr are connected, respectively, to the numerous (e.g. 1,024×1,024 or 4,096×4,096) carrier collecting electrodes 33 arranged in a two-dimensional matrix of rows and columns. Each set of carrier collecting electrode 33, capacitor Ca and thin-film transistor Tr acts as a separate detecting element DU. The voltage application electrode 32 is formed over the entire surface as a common electrode of all the detecting elements DU. As shown in FIG. 3, the data lines 34 form a plurality of columns juxtaposed in the horizontal (X) direction, while, as shown in FIG. 3, the gate lines 35 form a plurality of rows juxtaposed in the vertical (Y) direction. Each data line 34 and each gate line 35 are connected to the detecting elements DU. The data lines 34 are connected to an amplifier array circuit 37. The gate lines 35 are connected to a gate driver circuit 38. The number of detecting elements DU is not limited to 1,024×1,024 or 4,096×4,096, but is variable according to forms of implementation. Thus, only one detecting element DU may be provided.

The detecting elements DU are patterned in the two-dimensional matrix arrangement on the insulating substrate 36. The insulating substrate 36 having the detecting elements DU patterned thereon is also called “active matrix substrate”.

For forming the detecting elements DU and adjacent components of FPD 3, the data lines 34 and gate lines 35 are wired, and the thin-film transistors Tr, capacitors Ca, carrier collecting electrodes 33, thick semiconductor film 31 and voltage application electrode 32 are successively laminated on the surface of the insulating substrate 36 by using a thin-film formation technique based on a varied vacuum deposition method or photolithographic patterning. The semiconductor for forming the thick semiconductor film 31 may be selected appropriately according to use and withstand voltage, as exemplified by amorphous semiconductor and polycrystallized semiconductor.

The amplifier array circuit 37, including the analog-to-digital converter 8 disposed externally of the FPD 3, has a function to receive the carriers. That is, the analog-to-digital converter 8 and amplifier array circuit 37 read, through the detecting elements DU of the FPD 3, the carriers converted by the semiconductor thick film 31. The capacitors Ca correspond to the storage circuits in this invention. The analog-to-digital converter 8 and amplifier array circuit 37 correspond to the reading circuits in this invention. Thus, the image sensor S including the capacitors Ca, analog-to-digital converter 8 and amplifier array circuit 37 corresponds to the storage and reading circuit in this invention. The analog-to-digital converter 8 may be included in the construction of FPD 3. These gate driver circuit 38, amplifier array circuit 37 and analog-to-digital converter 8 are the peripheral circuits of the FPD 3.

In addition, the FPD 3 has a power source 39. In Embodiment 1, including also Embodiments 2-4 to follow, the power source 39 supplies electric power to the reading circuits, such as the amplifier array circuit 37 and analog-to-digital converter 8. The FPD 3, FPD controller 5 and analog-to-digital converter 8 constitute the image sensor S in FIG. 3.

Next, operation of the X-ray apparatus and flat panel X-ray detector (FPD) in Embodiment 1, including also Embodiments 2-4 to follow, will be described. Radiation to be detected is emitted in the state of a high bias voltage V_{A} (e.g. several hundred volts to several tens of kilovolts) being applied to the voltage application electrode 32. This bias voltage V_{A} is applied also under control of the FPD controller 5.

Carriers are generated by incidence of the radiation, and are stored as charge information in the charge-storing capacitors Ca. A gate line 35 is selected by a signal-fetching scan signal (i.e. gate driving signal) to the gate driver circuit 38. Further, the detecting elements DU connected to this gate line 35 are designated. The carriers (charges) stored in the capacitors Ca of the designated detecting elements DU are outputted to the data lines 34 via the thin-film transistors Tr turned on by the signal on the selected gate line 35.

The address of each detecting element DU is designated based on the signal fetching scan signals on the data line 34 and gate line 35 (i.e. the gate driving signal on the gate line 35 and amplifier driving signal on the data line 34). When a signal fetching scan signal is inputted to the amplifier array circuit 37 and gate driver circuit 38, each detecting element DU is selected by a scan signal (gate driving signal) of the vertical (Y) direction outputted from the gate driver circuit 38. Then, the amplifier array circuit 37 is switched by a scan signal of the horizontal (X) direction (amplifier driving signal), whereby the carriers (charges) from the capacitor Ca of a selected detecting element DU are outputted to the amplifier array circuit 37 through the data line 34. The charges are amplified by the amplifier array circuit 37, and outputted as X-ray detection signals from the amplifier array circuit 37 to the analog-to-digital converter 8.

Where the image sensor S having the FPD 3 in Embodiment 1 is used in the X-ray apparatus for detecting X-ray images, for example, the above operation causes the amplifier array circuit 37 to amplify charge information (X-ray detection signals) read through the data lines 34 as voltages, convert it to image information and output it as an X-ray image. Thus, the X-ray apparatus is constructed to obtain X-ray images based on the charge information (X-ray detection signals) stored in and read from the image sensor S including the capacitors Ca, analog-to-digital converter 8 and amplifier array circuit 37.

Next, the division of an image, setting for reading, and signals relating to each frame rate according to Embodiment 1, will be described with reference to FIGS. 4-6. FIG. 4 is a timing chart of frame rates and signals relating thereto according to Embodiment 1. FIG. 5 is a schematic view of an image divided into four. FIG. 6 shows timing charts of signals before and after the hand switch is depressed in the timing chart of FIG. 4.

As noted in the “Background Art” section, frame rates T are cycles relating to a series of operations for storage and reading of carriers. During these frame rates T, carriers for frames are read (see D1-D4 in FIG. 4). Times other than the image reading are the times when X-ray irradiation is enabled (X-ray irradiation enable times in FIG. 4). Specifically, as shown in FIG. 4, reading is started in the order of D1-D4 synchronously with a frame synchronization signal outputted for each frame rate T. Here, an X-ray image is divided equally into four areas D1, D2, D3 and D4 along the gate lines 35 as shown in FIG. 5. The controller 10 sets for the carriers to be read separately according to the divided areas D1-D4. Assuming that D1 is the first area and D4 the last area, when (carrier reading for) the last area D4 is finished, the operation returns to the first area D1 to repeat the reading. That is, the carriers are read periodically in the order of the divided adjoining areas, and when the last area D4 is finished, the operation returns to the first area D1 to repeat the reading.

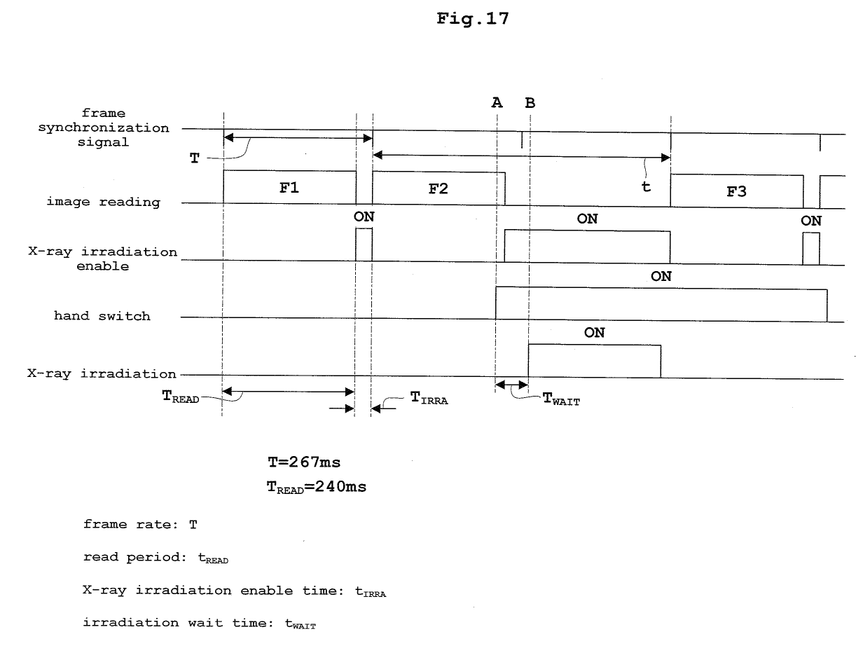

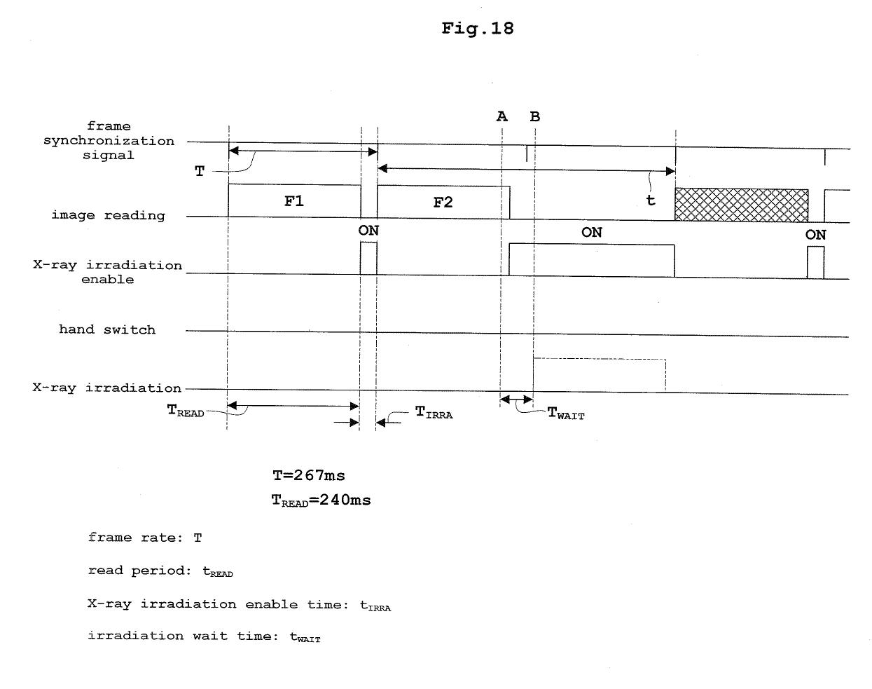

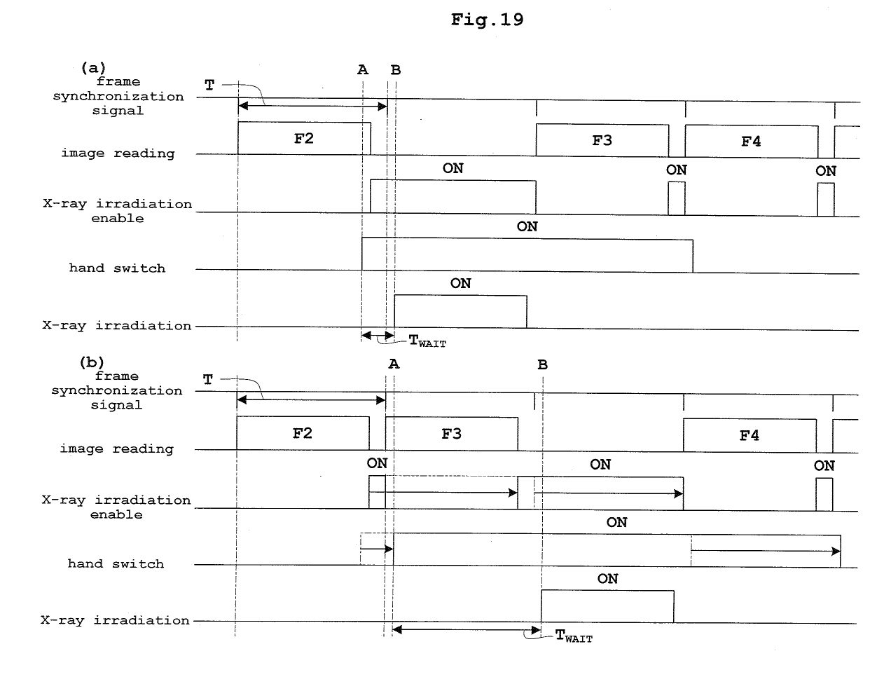

As shown in FIG. 4, assuming that the time of image reading for each frame rate T is a “read period”, the time other than the read period in each frame rate T is a time when X-ray irradiation is enabled. This time when X-ray irradiation is enabled is called “X-ray irradiation enable time”. Carrier storage for each frame is carried out in each frame rate T, including the read period and X-ray irradiation enable time. Assuming the read period to be t_{READ} and the X-ray irradiation enable time to be t_{IRRA}, T=t_{READ}+t_{IRRA} is established as clearly seen from the reason set out above. In FIG. 4, the read period t_{READ} is set to 60 ms, and the frame rate T to 66 ms. Conventionally, as shown in FIG. 17, the read period t_{READ} has been 240 ms, and the frame rate T 267 ms. The read period t_{READ} can be set as short as one fourth of that in the prior art, correspondingly to the division of an image into four areas (here, 240 ms×¼=60 ms). As a result, the frame rate T can also be reduced (here, from 267 ms to 66 ms).

In actually emitting X rays, X-ray pulses are emitted during the X-ray irradiation enable times. Also at non-irradiation times before start of X-ray irradiation, carriers are usually read for the divided areas as shown in FIG. 4, in order to release leak currents occurring at the non-irradiation times, and X-ray irradiation enable times t_{IRRA} are set as are irradiation times.

At a non-irradiation time before start of X-ray irradiation, the hand switch is depressed at time A in FIG. 4 to make a shift to preparation for the X-ray irradiation. The depression of the hand switch results in a shift to preparation for the X-ray irradiation. In the case of FIG. 4, the X-ray irradiation enable signal is continuously outputted without synchronizing with a first frame synchronization signal outputted after the shift to preparation for the X-ray irradiation, and is stopped synchronously with a frame synchronization signal outputted next. As a result, only for a period from time A to stopping of the X-ray irradiation enable signal, the X-ray irradiation enable time is longer than the usual X-ray irradiation enable time t_{IRRA}. X-ray pulses are emitted during this extended X-ray irradiation enable time.

Specifically, X-ray pulses are emitted at time B in FIG. 4, which is synchronized with the first frame synchronization signal outputted after the shift to preparation for X-ray irradiation, or synchronized with a predetermined time from this frame synchronization signal. The emission of X-ray pulses is stopped by the time the X-ray irradiation enable signal is stopped synchronously with the frame synchronization signal outputted next. Carriers are read for an area (see D3 in FIG. 4) immediately after the X-ray pulses are emitted, reading is carried out for the respective areas (see D4, D1 and D2 in FIG. 4) until the carriers are read for the entire area of the image, and imaging is carried out using the carriers read. The time from time A for depressing the hand switch to time B for outputting the X-ray pulses is called “irradiation wait time”. The irradiation wait time is referenced t_{WAIT}.

Since such hand switch is depressed manually, the shift to preparation for X-ray irradiation does not synchronize with a frame synchronization signal. Therefore, as shown in FIG. 6(a), for example, when the hand switch is depressed halfway through reading for area D2, the X-ray irradiation enable signal is continuously outputted without synchronizing with a first frame synchronization signal outputted immediately after the area D2, and is stopped synchronously with a frame synchronization signal outputted next. X-ray pulses are outputted at time B, which is synchronized with this frame synchronization signal, or synchronized with a predetermined time from the frame synchronization signal.

On the other hand, when, as shown in FIG. 6(b), the hand switch is depressed immediately after start of reading for the next area D3, the X-ray irradiation enable signal is continuously outputted without synchronizing with a first frame synchronization signal outputted immediately after the area D3, and is stopped synchronously with a frame synchronization signal outputted next. X-ray pulses are outputted at time B, which is synchronized with this frame synchronization signal, or synchronized with a predetermined time from the frame synchronization signal.

The irradiation wait time t_{WAIT} is variable up to a maximum corresponding to the frame rate T as shown in FIG. 6. In Embodiment 1, the variation is reduced to the frame rate T of 66 ms set as short as approximately one fourth of the variation corresponding to the frame rate T of 267 ms in the prior art.

With the X-ray apparatus according to Embodiment 1 described above, the controller 10 divides an image into a plurality of predetermined areas (divides an image equally into four areas D1-D4 in FIG. 5), and sets for reading of carriers before irradiation separately according to images of the divided areas (four areas D1-D4 in FIGS. 4 and 6). By dividing the reading of carriers before the irradiation in this way, when compared with the reading of carriers for all areas of an image (i.e. a frame) in the prior art, the read period t_{READ} or each frame rate T can be shortened to one of the number of divisions (one fourth in FIGS. 4-6). The time serving as the starting point of irradiation wait time t_{WAIT} (time A at which the hand switch is depressed in Embodiment 1) occurs before the reading of carriers before the irradiation. Even if the time serving as the starting point of irradiation wait time t_{WAIT} varies, the variation takes place only during each read period t_{READ} or each frame rate T set short. Therefore, the variation in irradiation wait time t_{WAIT} is made less than in the prior art, thereby improving response.

In FIG. 4, the controller 10 (see FIG. 1) sets for reading of carriers before the irradiation to be carried out periodically, and interposes, in an arbitrary cycle, a non-reading operation (X-ray irradiation enable in FIG. 4) between reading in that cycle and reading in the following cycle. Such controller 10 is applicable to an imaging apparatus which performs controls synchronously with cycles. In FIG. 4, the non-reading operation (X-ray irradiation enable) may be set to the storage of carriers, or the reading and the non-reading operation (X-ray irradiation enable) may be set to the storage of carriers.

In FIG. 4, the controller 10 (see FIG. 1) sets for stopping the reading of carriers before the irradiation, even if the reading of carriers before the irradiation is only halfway through an image, according to the image of a divided area (D2 in FIG. 4) corresponding to the halfway area, and synchronously with the cycle corresponding to the halfway timing. The X-ray tube controller 7 (see FIG. 1) controls the X-ray tube 2 to emit X rays after the reading of carriers before the irradiation is stopped by the controller 10 and at the time of non-reading operation (X-ray irradiation enable).

With such X-ray tube controller 7 provided, the reading of carriers before the irradiation can be stopped by the controller 10, even if the reading of carriers before the irradiation is only halfway through an image, according to the image of a divided area (D2 in FIG. 4) corresponding to the halfway area. And, the X-ray tube controller 7 controls the X-ray tube 1 (see FIG. 1) to emit X rays after the reading of carriers before the irradiation is stopped by the controller 10 and at the time of non-reading operation (X-ray irradiation enable). Thus, X rays can be emitted even if the reading of carriers before the irradiation is only halfway through an image.

In FIG. 4, the reading of carriers before the irradiation is carried out periodically in the order of divided adjoining areas (the order of D1, D2, D3 and D4 in FIG. 4), and when carrier reading for the last area (D4 in FIG. 4) is finished, the operation returns to the first area (D1 in FIG. 4) to repeat the reading. The carriers can be read before the irradiation in this way.

In FIG. 4, the reading of carriers at an irradiation time is started from the next area (D3 in FIG. 4) adjacent the area (D2 in FIG. 4) where the above carrier reading is stopped. The reading of carriers at the irradiation time is carried out periodically in the order of the divided adjoining area (the order of D4 in FIG. 4) from the starting area (D3 in FIG. 4). When the last area (D4 in FIG. 4) is finished, the operation returns to the first area (D1 in FIG. 4) to repeat the reading.

In this way, the reading of carriers at the irradiation time can be started from the next area (D3 in FIG. 4) adjacent the area (D2 in FIG. 4) where the carrier reading is stopped. Even if the next area (D3 in FIG. 4) which is the area where the reading of carriers at the irradiation time is started is not the first area (D1 in FIG. 4) as shown in FIG. 4, the operation returns to the first area (D1 in FIG. 4) when the last area (D4 in FIG. 4) is finished, to repeat the reading. Thus, the reading of carriers at the irradiation time can be carried out over all areas. That is, image reading can be carried out over all the areas of the image, for imaging.

In Embodiment 1, including also Embodiments 2-4 to follow, the image processor 9 (see FIG. 1) has a function to correct the carriers read at the irradiation time, based on the carriers read at the non-irradiation time. This invention can be applied where charge information is corrected (dark correction) based on the carriers read at the non-irradiation time (dark image information). The image processor 9 corresponds to the correcting device in this invention.

In Embodiment 1, including also Embodiments 2-4 to follow, carriers are read before the irradiation as noted above, that is leak currents are read beforehand, and the leak currents read are once stored and written as dark image information in the memory 11 (see FIG. 1) through the analog-to-digital converter 8, image processor 9 and controller 10 (see FIG. 1 for all). Subsequently, the carriers read at the irradiation time are once stored and written as X-ray detection signals in the memory 11 (see FIG. 1) through the analog-to-digital converter 8, image processor 9 and controller 10 (see FIG. 1 for all). At a time of dark correction by the image processor 9, the dark image information and X-ray detection signals written in the memory 11 are read, the dark correction is carried out through a correction process such as of subtracting the dark image information from the X-ray detection signals, and the data after the dark correction is once stored and written as an X-ray image in the memory 11. This X-ray image after the dark correction is outputted to and displayed on the monitor 13 (see FIG. 1), for example. To summarize the above, the dark image information read at the non-irradiation time and used in the correction consists of the carriers read before the irradiation in Embodiment 1.

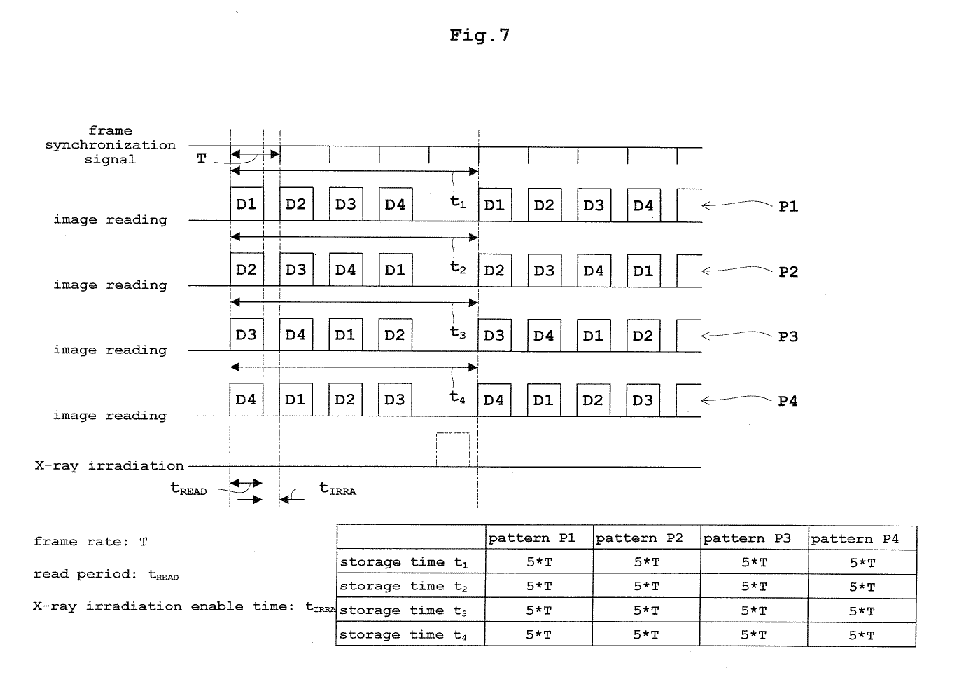

The case of applying this dark image information to the timing chart of FIG. 4 as in Embodiment 1 will be described with reference to FIG. 7. FIG. 7 is an explanatory view of the dark image information according to Embodiment 1. In carrying out the dark correction, as shown in FIG. 7, the carriers for areas read with the same timing as in FIG. 4 and without outputting X-ray pulses are read as carriers at the X-ray non-irradiation time. The dark correction is carried out by using the carriers read as dark image information.

In FIG. 4, the reading of carriers before the irradiation is carried out periodically in the order of divided adjoining areas (the order of D1, D2, D3 and D4 in FIG. 4), and when carrier reading for the last area (D4 in FIG. 4) is finished, the operation returns to the first area (D1 in FIG. 4) to repeat the reading. And, even if the reading of carriers before the irradiation is only halfway through an image, the reading of carriers before the irradiation is stopped according to the image of a divided area corresponding to the halfway area. And, the reading of carriers at the irradiation time is started from the next area adjacent the area where the carrier reading is stopped. The reading of carriers at the irradiation time is carried out periodically in the order of the divided adjoining areas from the starting area. When the last area is finished, the operation returns to the first area to repeat the reading. Therefore, when an image is divided equally into four areas D1-D4, the reading takes four patterns P1, P2, P3 and P4 as shown in FIG. 7.

Specifically, in pattern P1, carriers are read before the irradiation in the order of areas D1, D2, D3 and D4, and carriers are read at the irradiation time in the order of areas D1, D2, D3 and D4. In pattern P2, carriers are read before the irradiation in the order of areas D2, D3, D4 and D1, and carriers are read at the irradiation time in the order of areas D2, D3, D4 and D1. In pattern P3, carriers are read before the irradiation in the order of areas D3, D4, D1 and D2, and carriers are read at the irradiation time in the order of areas D3, D4, D1 and D2. In pattern P4, carriers are read before the irradiation in the order of areas D4, D1, D2 and D3, and carriers are read at the irradiation time in the order of areas D4, D1, D2 and D3.

A storage time from start of a previous area identical to an area to be imaged to start of the area to be imaged is referred to as ti for area D1, t₂ for area D2, t₃ for area D3 and t₄ for area D4 (see FIGS. 4 and 7). Storage times t₁, t₂, t₃ and t₄ for the respective areas, when expressed in terms of frame rate T, are t₁=t₂=t₃=t₄=5×T in all patterns P1-P4, as shown in FIG. 7.

The characteristics of dark image information will change depending on the length of storage times t₁, t₂, t₃ and t₄. Thus, when, as shown in FIG. 4, images are processed by carrying out the reading of carriers before the irradiation in the order of areas D3, D4, D1 and D2 and the reading of carries at the irradiation time in the order of areas D3, D4, D1 and D2, the imaging will be in a pattern corresponding to pattern P3. Normally, it is ideal to carry out the dark correction using the carriers (dark image information) read before the irradiation in pattern P3. However, since t₁=t₂=t₃=t₄=5×T applies commonly to all patterns P1-P4 in Embodiment 1 as noted above, the dark correction may be carried out using the carriers (dark image information) read before the irradiation in a pattern applicable any one of the patterns P1-P4.

To summarize the above, in Embodiment 1, the reading of carriers before the irradiation is carried out periodically in the order of the divided adjoining areas, and when the carrier reading for the last area is finished, the operation returns to the first area to repeat the reading. Even if the reading of carriers before the irradiation is only halfway through an image, the reading of carriers before the irradiation is stopped according to the image of a divided area corresponding to the halfway area. The reading of carriers at the irradiation time is started from the next area adjacent the area where the carrier reading is stopped. The reading of carriers at the irradiation time is carried out periodically in the order of the divided adjoining areas from the starting area. When the last area is finished, the operation returns to the first area to repeat the reading. The dark correction can be carried out accurately only with one piece of dark image information.

### Embodiment 2

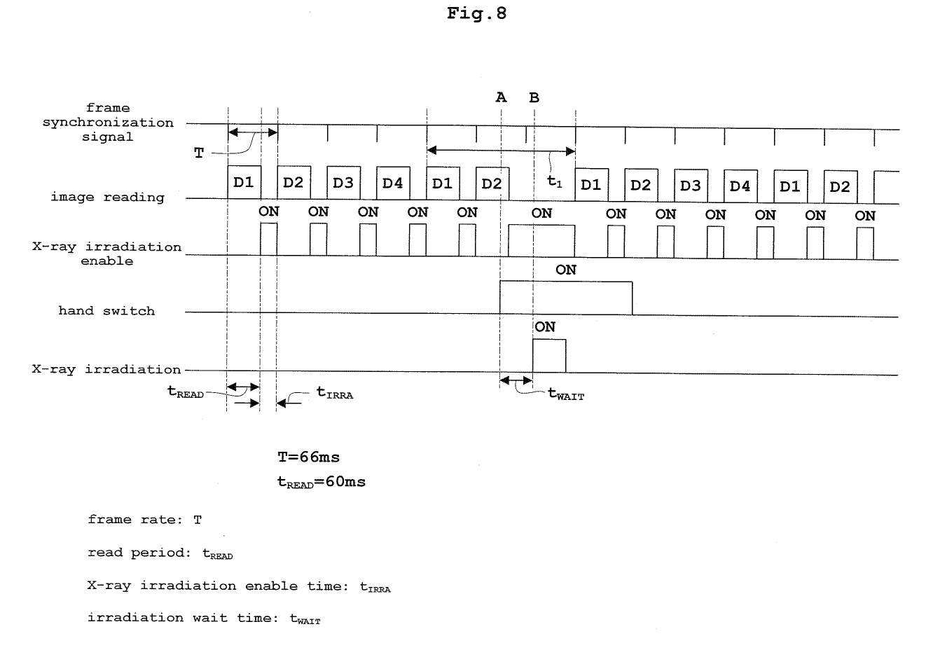

Next, Embodiment 2 of this invention will be described with reference to the drawings. FIG. 8 is a timing chart of frame rates and signals relating thereto according to Embodiment 2. The X-ray apparatus and FPD in Embodiment 2 have the same constructions as in Embodiment 1 described above. Thus, their description will be omitted, and only the difference will be described.

The difference to Embodiment 1 lies in that the controller 10 (see FIG. 1) has an area changing function for changing areas where the reading of carriers at the irradiation time is started. However, as in Embodiment 1, the reading of carriers at the irradiation time is carried out periodically in the order of the divided adjoining areas from the starting area, and when the last area (D4 in FIG. 8) is finished, the operation returns to the first area (D1 in FIG. 8) to repeat the reading. The following description will be made on an assumption that, in Embodiment 2, the reading of carriers at the irradiation time is started from the first area (D1 in FIG. 8). The controller 10 in Embodiment 2 corresponds to the first storage and readout setting device, the second readout setting device, the readout stopping device and the area changing device in this invention.

Specifically, as shown in FIG. 8, the reading of carriers before the irradiation is carried out in the order of areas D3, D4, D1 and D2, and the area for starting the reading of carriers at the irradiation time is changed to the first area D1. And the reading of carriers at the irradiation time is carried out periodically in the order of the divided adjoining areas (D2, D3 and D4 in FIG. 8) from the starting area D1.

With the X-ray apparatus according to Embodiment 2 described above, the controller 10 changes the area for starting the reading of carriers at the irradiation time, and thus it is possible to select an arbitrary area for starting the reading of carriers at the irradiation time. In Embodiment 2, the arbitrary area for starting the reading of carriers at the irradiation time is the first area (D1 in FIG. 8). Even if this is not the first area, the reading of carriers at the irradiation time can be carried out over all areas since the operation returns to the first area (D1 in FIG. 8) when the last area (D4 in FIG. 8) is finished, to repeat the reading.

In Embodiment 2, the area for starting the reading of carriers at the irradiation time is changed to the first area, such that the reading of carriers at the irradiation time is started from the first area (D1 in FIG. 8). However, as noted above, the area for starting the reading of carriers at the irradiation time is not limited to the first area, but may be any arbitrary area.

Where, as in Embodiment 1 described hereinbefore, the reading of carriers at the irradiation time is started from the next area (D3 in FIG. 4) adjacent the area (D2 in FIG. 4) where the carrier reading has stopped, a luminance difference occurs between area D2 and area D3 divided when reading carriers. In Embodiment 2, the luminance difference occurring at the boundary between divided images when reading carriers from such intermediate areas can be solved by starting the reading of carriers at the irradiation time from the first area.

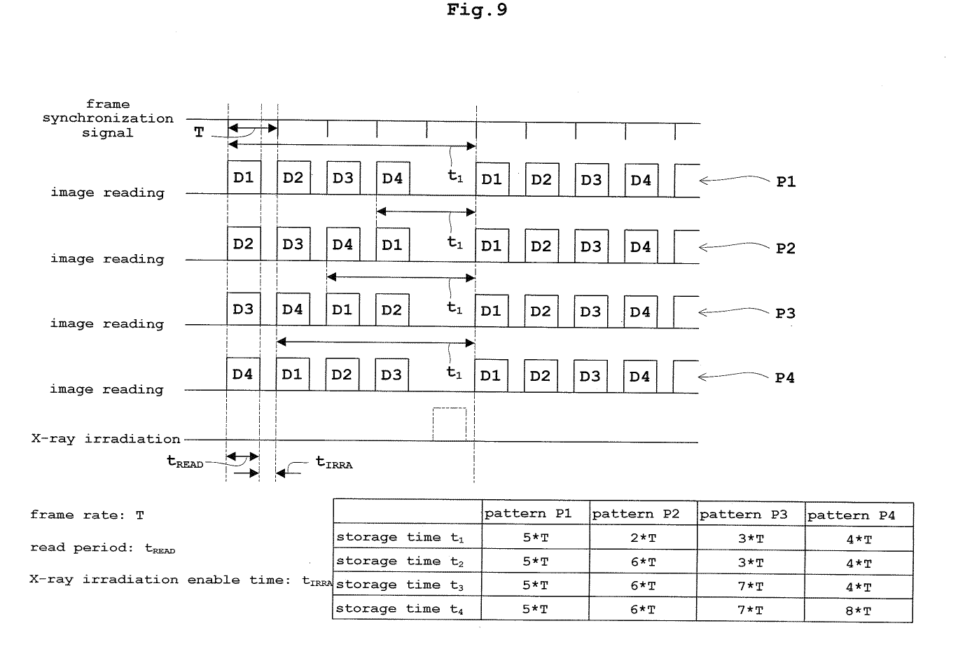

The case of applying the dark image information to the timing chart of FIG. 8 as in Embodiment 2 will be described with reference to FIG. 9. FIG. 9 is an explanatory view of the dark image information according to Embodiment 2. In carrying out a dark correction, as shown in FIG. 9, the carriers for areas read with the same timing as in FIG. 8 and without outputting X-ray pulses are read as carriers at the X-ray non-irradiation time. The dark correction is carried out by using the carriers read as dark image information. In this case, the reading takes four patterns P1, P2, P3 and P4 as shown in FIG. 9.

Specifically, in pattern P1, carriers are read before the irradiation in the order of areas D1, D2, D3 and D4, and carriers are read at the irradiation time in the order of areas D1, D2, D3 and D4. In pattern P2, carriers are read before the irradiation in the order of areas D2, D3, D4 and D1, and carriers are read at the irradiation time in the order of areas D1, D2, D3 and D4. In pattern P3, carriers are read before the irradiation in the order of areas D3, D4, D1 and D2, and carriers are read at the irradiation time in the order of areas D1, D2, D3 and D4. In pattern P4, carriers are read before the irradiation in the order of areas D4, D1, D2 and D3, and carriers are read at the irradiation time in the order of areas D1, D2, D3 and D4.

As in Embodiment 1, a storage time from start of a previous area, identical to an area to be imaged, to start of the area to be imaged is referred to as t₁ for area D1, t₂ for area D2, t₃ for area D3 and t₄ for area D4. Storage times ti, t₂, t₃ and t₄ for the respective areas, when expressed in terms of frame rate T, are as follows, as shown in FIG. 9.

In pattern P1, t₁=5×T, t₂−5×T, t₃=5×T and t₄=5×T. In pattern P2, t₁−2×T, t₂×6×T, t₃=6×T and t₄−6×T. In pattern P3, t₁−3×T, t₂=3×T, t₃=7×T and t₄−7×T. In pattern P4, t₁=4×T, t₂=4×T, t₃=4×T and t₄−8×T.

As noted in Embodiment 1, the characteristics of dark image information will change depending on the length of storage times t₁, t₂, t₃ and t₄. Thus, when, as shown in FIG. 8, images are processed by carrying out the reading of carriers before the irradiation in the order of areas D3, D4, D1 and D2, and the area for starting the reading of carriers at the irradiation time is changed to area D1 to carry out the reading of carries at the irradiation time in the order of areas D1, D2, D3 and D4, the images will be in a pattern corresponding to pattern P3. Thus, it is ideal carry out the dark correction using the carriers (dark image information) read before the irradiation in pattern P3.

In Embodiment 2, as distinct from Embodiment 1 described hereinbefore, the storages times t₁, t₂, t₃ and t₄ for the respective patterns P1-P4 are different from one another, and the characteristics of the dark image information are different. It is therefore desirable to have plural pieces of dark image information for the respective patterns (four patterns P1-P4 in this case). The start of irradiation is determined by the time of stopping the storage and reading or stopping the reading (which is time A of depressing the hand switch in this case), and the time of starting irradiation is unknown. That is, depending on the time, imaging can be in a pattern corresponding to each of patterns P1-P4. Therefore, considering that the time of starting irradiation is unknown, plural pieces of dark image information matched with times of starting irradiation are provided, thereby carrying out the dark correction with increased accuracy.

### Embodiment 3

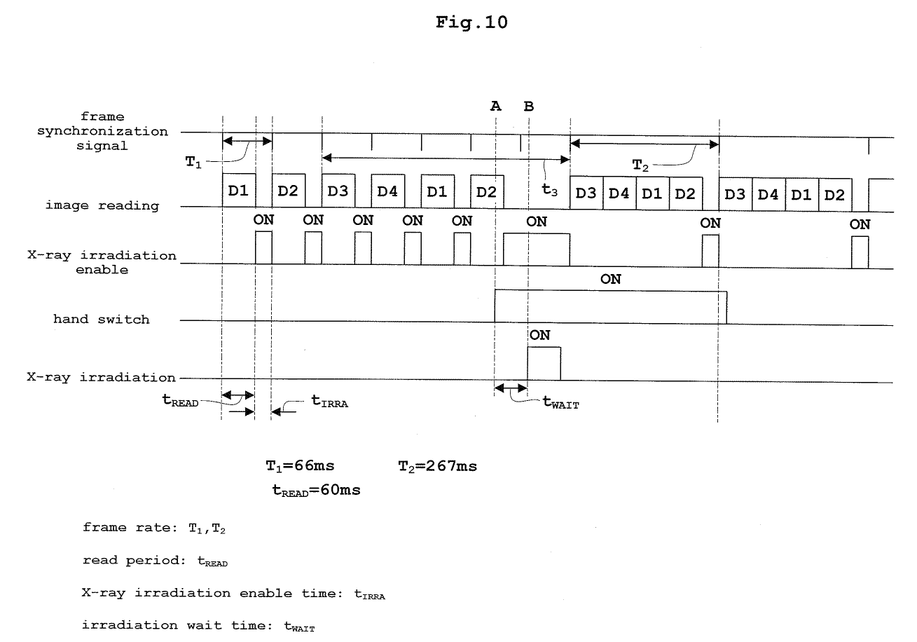

Next, Embodiment 3 of this invention will be described with reference to the drawings. FIG. 10 is a timing chart of frame rates and signals relating thereto according to Embodiment 3. The X-ray apparatus and FPD in Embodiment 3 have the same constructions as in Embodiments 1 and 2 described above. Thus, their description will be omitted, and only the difference will be described.

The difference to Embodiments 1 and 2 lies in that the reading of carriers at the irradiation time is carried out continuously according to all the areas of an image. The reading of carriers before the irradiation is the same as in Embodiments 1 and 2 in that it is carried out periodically in the order of the divided adjoining areas (in the order of D1, D2, D3 and D4 in FIG. 10), and when the reading of carriers for the last area (D4 in FIG. 10) is finished, the operation returns to the first area (D1 in FIG. 10) to repeat the reading.

Specifically, as shown in FIG. 10, the reading of carriers before the irradiation is carried out in the order of areas D3, D4, D1 and D2, and the reading of carriers at the irradiation time is carried out continuously according to all the areas of the image. This is carried out continuously in the order of areas D3, D4, D1 and D2. Consequently, compared with the frame rate before the irradiation, the frame rate after the irradiation becomes long as does the frame rate in the prior art. When the frame rate before the irradiation is T₁ and the frame rate after the irradiation is T₂, T₁ becomes 66 ms, and T₂ 267 ms.

With the X-ray apparatus according to Embodiment 3 described above, the reading of carriers at the irradiation time is carried out continuously according to all the areas of an image, whereby the reading is carried out faster than the carrier reading before the irradiation. In the case of FIG. 10, since the X-ray irradiation enable times are omitted during the irradiation, the reading can be carried out at a correspondingly increased speed. As long as the storage and reading of carriers before the irradiation are carried out separately in this invention, the question of response which is the problem addressed by this invention can be solved. In Embodiment 3 and in Embodiment 4 to follow, the reading of carriers at the irradiation time may be carried out continuously according to all the areas of an image.

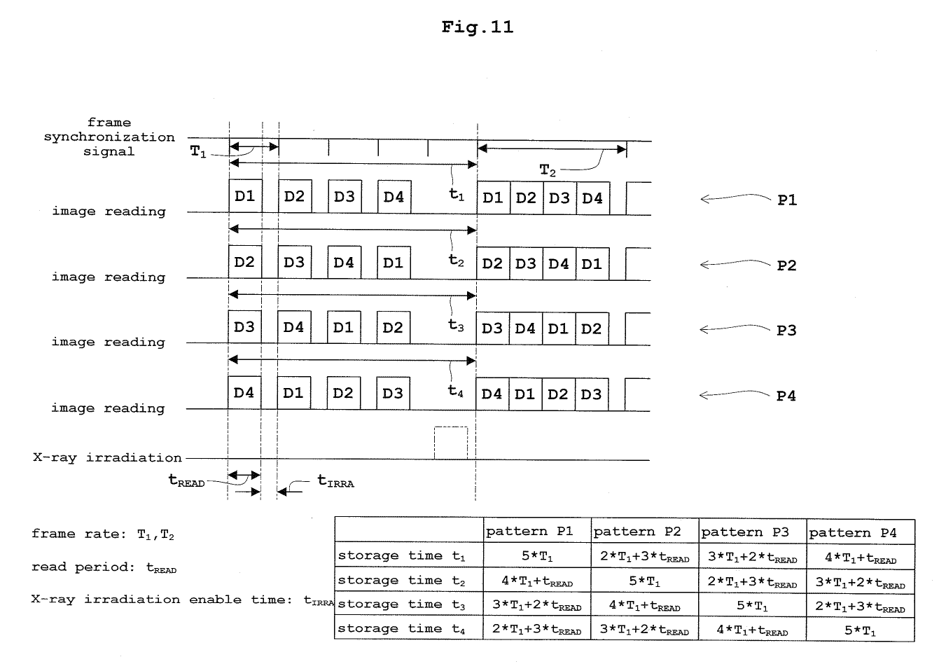

The case of applying the dark image information to the timing chart of FIG. 10 as in Embodiment 3 will be described with reference to FIG. 11. FIG. 11 is an explanatory view of the dark image information according to Embodiment 3. In carrying out a dark correction, as shown in FIG. 11, the carriers for the areas read with the same timing as in FIG. 10 and without outputting X-ray pulses are read as carriers at the X-ray non-irradiation time. The dark correction is carried out by using the carriers read as dark image information. In this case, the reading takes four patterns P1, P2, P3 and P4 as shown in FIG. 11.

Specifically, in pattern P1, carriers are read before the irradiation separately in the order of areas D1, D2, D3 and D4, and carriers are read at the irradiation time continuously in the order of areas D1, D2, D3 and D4. In pattern P2, carriers are read before the irradiation separately in the order of areas D2, D3, D4 and D1, and carriers are read at the irradiation time continuously in the order of areas D2, D3, D4 and D1. In pattern P3, carriers are read before the irradiation separately in the order of areas D3, D4, D1 and D2, and carriers are read at the irradiation time in the order of areas D3, D4, D1 and D2. In pattern P4, carriers are read before the irradiation separately in the order of areas D4, D1, D2 and D3, and carriers are read at the irradiation time continuously in the order of areas D4, D1, D2 and D3.

As in Embodiments 1 and 2, a storage time from start of a previous area, identical to an area to be imaged, to start of the area to be imaged is referred to as t₁ for area D1, t₂ for area D2, t₃ for area D3 and t₄ for area D4. Storage times t₁, t₂, t₃ and t₄ for the respective areas, when expressed in terms of frame rate T and read period t_{READ}, are as follows, as shown in FIG. 11.

In pattern P1, t₁=5×T₁, t₂=4×T₁+t_{READ}, t₃=3×T₁+2×t_{READ} and t₄=2×T₁+3×t_{READ}. In pattern P2, t₁=2×T₁+3×t_{READ}, t₂−5×T₁, t₃=4×T₁+t_{READ} and t₄=3×T₁+2>t_{READ}. In pattern P3, t₁=3×T₁+2×t_{READ}, t₂=2×T₁+3×t_{READ}, t₃=5×T₁ and t₄−4×T₁+t_{READ}. In pattern P4, t₁=4×T₁+t_{READ}, t₂=3×T₁+2×t_{READ}, t₃=2×T₁+3×t_{READ} and t₄=5×T₁.

As noted in Embodiments 1 and 2, the characteristics of dark image information will change depending on the length of storage times t₁, t₂, t₃ and t₄. Thus, when, as shown in FIG. 10, images are processed by carrying out the reading of carriers before the irradiation separately in the order of areas D3, D4, D1 and D2, and the reading of carries at the irradiation time is carried out continuously in the order of areas D3, D4, D1 and D2, the imaging will be in a pattern corresponding to pattern P3. Thus, it is ideal carry out the dark correction using the carriers (dark image information) read before the irradiation in pattern P3.

In Embodiment 3, as distinct from Embodiment 1 described hereinbefore, and as in Embodiment 2, the storages times t₁, t₂, t₃ and t₄ for the respective patterns P1-P4 are different from one another, and the characteristics of the dark image information are different. It is therefore desirable to have plural pieces of dark image information for the respective patterns (four patterns P1-P4 in this case).

### Embodiment 4

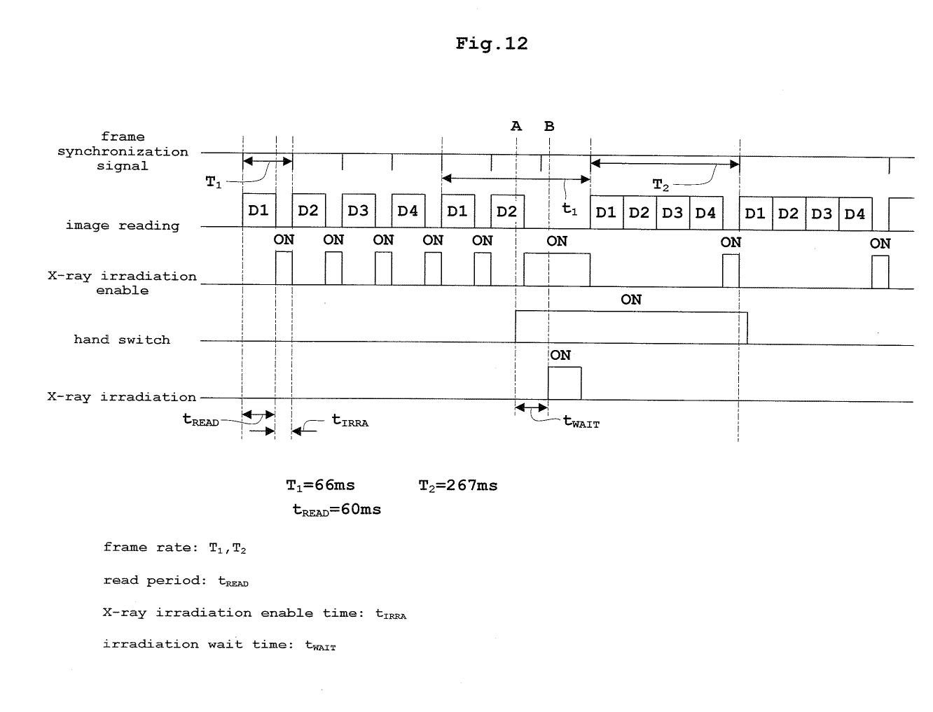

Next, Embodiment 4 of this invention will be described with reference to the drawings. FIG. 12 is a timing chart of frame rates and signals relating thereto according to Embodiment 4. The X-ray apparatus and FPD in Embodiment 4 also have the same constructions as in Embodiments 1-3 described above. Thus, their description will be omitted, and only the difference will be described.

The difference to Embodiment 3 lies in that the controller 10 (see FIG. 1), as in Embodiment 2, has an area changing function for changing areas where the reading of carriers at the irradiation time is started. It is the same as in Embodiment 3 that the reading of carriers at the irradiation time is carried out continuously according to all the areas of an image. That is, Embodiment 4 is an implementation mode combining Embodiment 2 and Embodiment 3.

Specifically, as shown in FIG. 12, the reading of carriers before the irradiation is carried out in the order of areas D3, D4, D1 and D2, and the reading of carriers at the irradiation time is carried out continuously according to all the areas of the image. This is carried out continuously in the order of areas D1, D2, D3 and D4. As in Embodiment 3, the frame rate before the irradiation is T₁ and the frame rate after the irradiation is T₂.

With the X-ray apparatus according to Embodiment 4 described above, as in Embodiment 3, the reading of carriers at the irradiation time is carried out continuously according to all the areas of an image, whereby the reading is carried out faster than the carrier reading before the irradiation. As in Embodiment 2, the luminance difference occurring at the boundary between divided images when reading carriers from intermediate areas can be solved by starting the reading of carriers at the irradiation time from the first area (D1 in FIG. 12).

In Embodiment 4, as in Embodiment 2 described hereinbefore, the area for starting the reading of carriers at the irradiation time is changed to the first area, such that the reading of carriers at the irradiation time is started from the first area (D1 in FIG. 12). However, as noted above, the area for starting the reading of carriers at the irradiation time is not limited to the first area, but may be any arbitrary area.

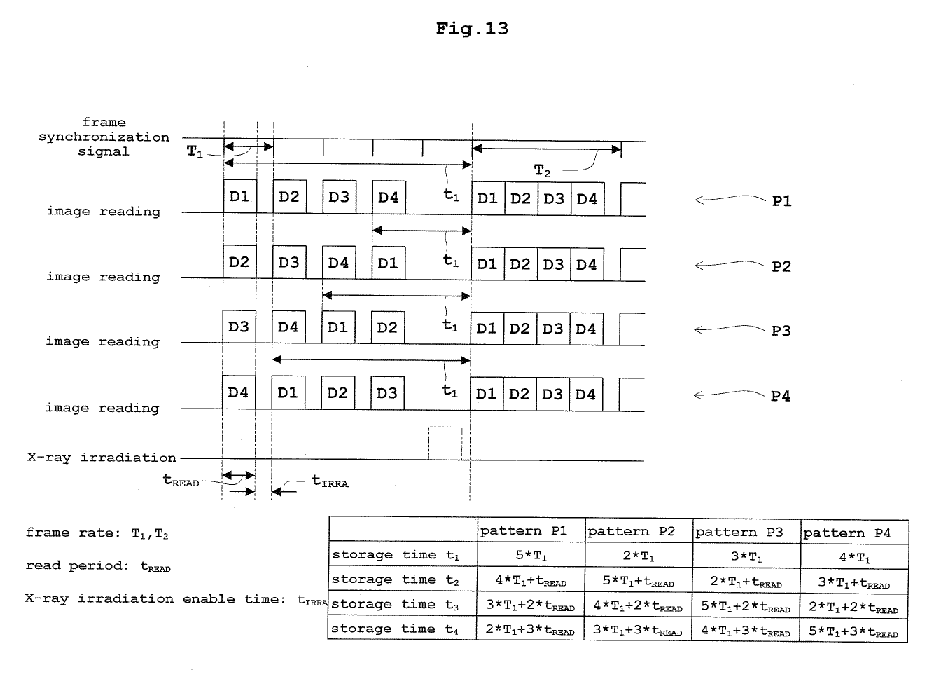

The case of applying the dark image information to the timing chart of FIG. 12 as in Embodiment 4 will be described with reference to FIG. 13. FIG. 13 is an explanatory view of the dark image information according to Embodiment 4. In carrying out a dark correction, as shown in FIG. 13, the carriers for areas read with the same timing as in FIG. 12 and without outputting X-ray pulses are read as carriers at the X-ray non-irradiation time. The dark correction is carried out by using the carriers read as dark image information. In this case, the reading takes four patterns P1, P2, P3 and P4 as shown in FIG. 13

Specifically, in pattern P1, carriers are read before the irradiation separately in the order of areas D1, D2, D3 and D4, and carriers are read at the irradiation time continuously in the order of areas D1, D2, D3 and D4. In pattern P2, carriers are read before the irradiation separately in the order of areas D2, D3, D4 and D1, and carriers are read at the irradiation time continuously in the order of areas D1, D2, D3 and D4. In pattern P3, carriers are read before the irradiation separately in the order of areas D3, D4, D1 and D2, and carriers are read at the irradiation time in the order of areas D1, D2, D3 and D4. In pattern P4, carriers are read before the irradiation separately in the order of areas D4, D1, D2 and D3, and carriers are read at the irradiation time continuously in the order of areas D1, D2, D3 and D4.

As in Embodiments 1-3, a storage time from start of a previous area, identical to start of an area to be imaged, to start of the area to be imaged is referred to as t₁ for area D1, t₂ for area D2, t₃ for area D3 and t₄ for area D4. Storage times t₁, t₂, t₃ and t₄ for the respective areas, when expressed in terms of frame rate T and read period t_{READ}, are as follows, as shown in FIG. 13.

In pattern P1, t₁−5×T₁, t₂=4×T₁+t_{READ}, t₃−3×T₁+2×t_{READ} and t₄=2×T₁+3×t_{READ}. In pattern P2, t₁=2×T₁, t₂=5×T₁+t_{READ}, t₃=4×T₁+2×t_{READ} and t₄=3×T₁+3×t_{READ}. In pattern P3, t₁=3×T₁, t₂=2×T₁+t_{READ}, t₃=5×T₁+2×t_{READ} and t₄−4×T₁+3×t_{READ}. In pattern P4, t₁=4×T₁, t₂=3×T₁+t_{READ}, t₃=2×T₁+2×t_{READ} and t₄=5×T₁+3×t_{READ}.

As in Embodiments 1-3, the characteristics of dark image information will change depending on the length of storage times t₁, t₂, t₃ and t₄. Thus, when, as shown in FIG. 12, images are processed by carrying out the reading of carriers before the irradiation separately in the order of areas D3, D4, D1 and D2, and the reading of carries at the irradiation time is carried out continuously in the order of areas D1, D2, D3 and D4, the imaging will be in a pattern corresponding to pattern P3. Thus, it is ideal carry out the dark correction using the carriers (dark image information) read before the irradiation in pattern P3.

In Embodiment 4, as distinct from Embodiment 1 described hereinbefore, and as in Embodiments 2 and 3, the storages times t₁, t₂, t₃ and t₄ for the respective patterns P1-P4 are different from one another, and the characteristics of the dark image information are different. It is therefore desirable to have plural pieces of dark image information for the respective patterns (four patterns P1-P4 in this case).

This invention is not limited to the foregoing embodiments, but may be modified as follows:

(1) In each of the foregoing embodiments, the X-ray apparatus shown in FIG. 1 has been described by way of example. This invention may be applied also to an X-ray apparatus mounted on a C-shaped arm, for example. This invention may be applied also to an X-ray fluoroscopic apparatus and an X-ray CT apparatus.

(2) In each of the foregoing embodiments, the invention is applied to a radiation detector of the “direct conversion type” with the thick semiconductor film 31 (semiconductor layer) converting incident radiation directly to charge information. The invention is applicable also to a radiation detector of the “indirect conversion type” with a converting layer such as a scintillator converting incident radiation into light, and a semiconductor layer formed of a light sensitive material converting the light to charge information. The light sensitive semiconductor layer may be formed of photodiodes.

(3) In each of the foregoing embodiments, the X-ray detector for detecting X rays has been described by way of example. This invention is not limited to a particular type of radiation detector which may, for example, be a gamma-ray detector for detecting gamma rays emitted from a patient dosed with radioisotope (RI), such as in an ECT (Emission Computed Tomography) apparatus. Similarly, this invention is applicable to any imaging apparatus that detects radiation, as exemplified by the ECT apparatus noted above.

(4) In each of the foregoing embodiments, the radiation detector for detecting radiation, typically X rays, has been described by way of example. This invention is applicable also to a photodetector for detecting light. Thus, the invention is not limited to any device that forms images by detecting light.

(5) Each of the foregoing embodiments is an implementation mode based on carrier reading which carries out the reading of carriers before the irradiation separately according to the images of divided areas, but may be an implementation mode based on the storage of carriers instead. That is, the storage of carriers may be carried out separately according to the images of divided areas. Then, the controller 10 (see FIG. 1) will have a storage and readout stopping function to stop the storage and reading of carriers before the irradiation. The controller 10 corresponds to the storage and readout stopping device in this invention.

(6) In each of the foregoing embodiments, the setting is made to read carriers before the irradiation periodically, and interpose, in an arbitrary cycle, a non-reading operation between reading in that cycle and reading in the following cycle. However, it is not absolutely necessary to synchronize it with the cycles. In this case, the controller 10 (see FIG. 1) may have a storage and readout stopping function to stop the storage and reading of carriers before the irradiation, even if the storage and reading of carriers before the irradiation is only halfway through an image, according to the image of a divided area corresponding to the halfway area, and the X-ray tube controller 7 (see FIG. 1) may have a function to control to emit X rays after the storage and reading of carriers before the irradiation is stopped by the storage and readout stopping function of the controller 10. The controller 10 corresponds to the storage and readout stopping device in this invention.

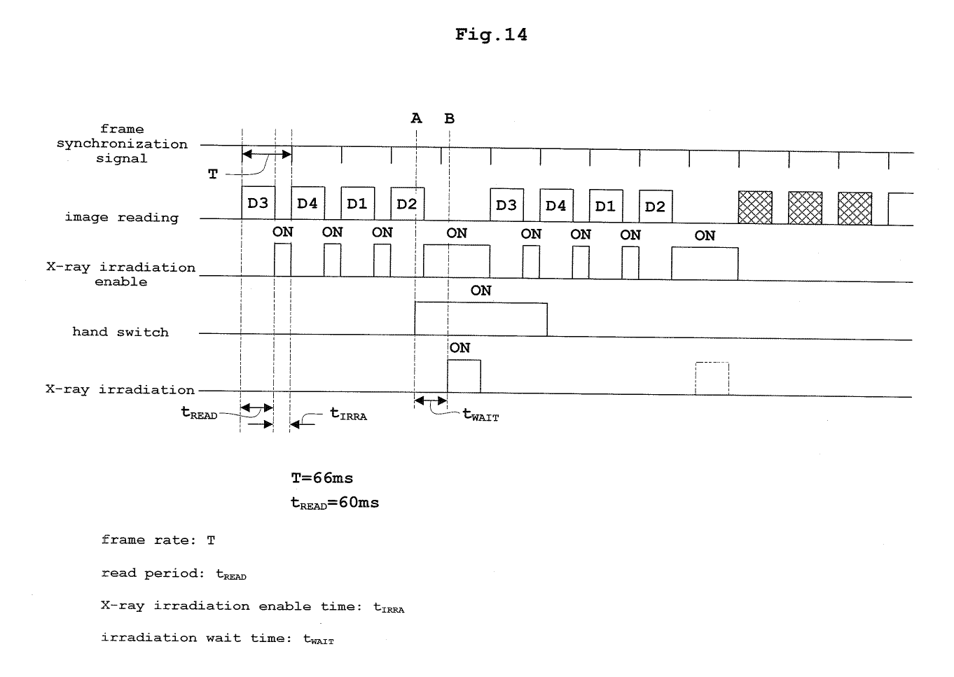

(7) In each of the foregoing embodiments, the dark image information read at the non-irradiation time for use in the correction consists of the carriers read before the irradiation. As shown in FIG. 14, this may be carriers read after the irradiation. In this case, a dark correction is carried out using as dark image information the carriers for areas (see the hatched areas in FIG. 14) read after imaging with the same timing as the imaging and without outputting X-ray pulses.

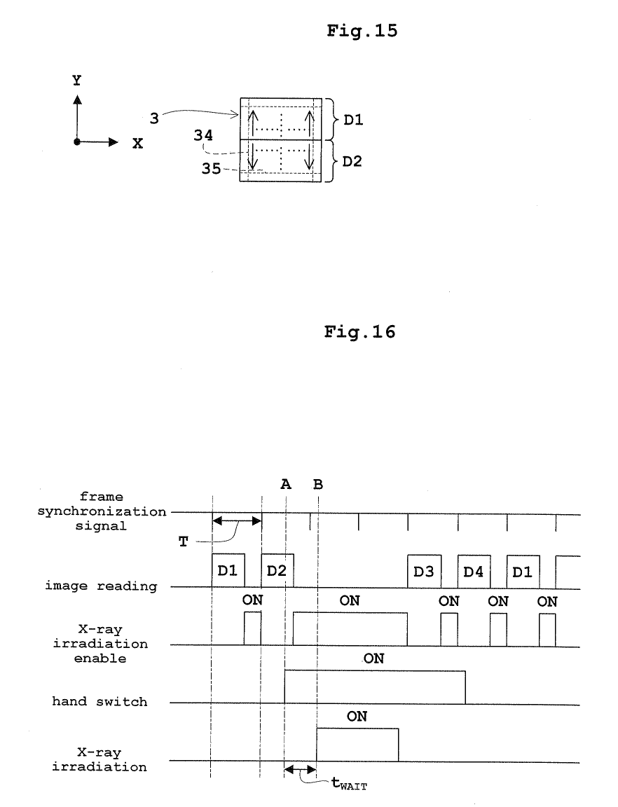

(8) In each of the foregoing embodiments, the image dividing mode is as shown in FIG. 5, but this is not limitative. As shown in FIG. 15, for example, it may be divided vertically into two equal parts. This case is useful particularly to an FPD that reads independently upward or downward through the data lines 34. The image may be divided laterally along the data lines 34.

(9) This invention is applicable to both a method of reading one line at a time through the data lines, and a method of reading by a plurality of lines through the data lines.

(10) In each of the foregoing embodiments, the X-ray irradiation enable signal is continuously outputted without synchronizing with a first frame synchronization signal outputted after a shift to preparation for X-ray irradiation, and is stopped synchronously with a frame synchronization signal outputted next, but this is not limitative. As shown in FIG. 16, the X-ray irradiation enable signal may be continuously outputted without synchronizing with the next frame synchronization signal, to be stopped synchronously with a frame synchronization signal outputted further next, thereby extending the X-ray irradiation enable time to set the emission of X-ray pulses long. Thus, by increasing the periodic number of the frame synchronization signal with which stopping of the X-ray irradiation signal is synchronized, the X-ray irradiation enable time can be extended further to set the emission of X-ray pulses to be still longer.

1. An imaging apparatus for obtaining images by carrying out imaging based on light or radiation, comprising a conversion layer for converting light or radiation information to charge information in response to incident light or radiation, and a storage and reading circuit for storing and reading the charge information converted by the conversion layer, the apparatus being constructed to obtain the images based on the charge information read by the storage and reading circuit, the apparatus further comprising a first storage and readout setting device for dividing an image into a plurality of predetermined areas, and setting storage and reading of the charge information before irradiation of the light or radiation according images of the divided areas.

2. The imaging apparatus according to claim 1, comprising a storage and readout stopping device for stopping the storage and reading of the charge information before the irradiation, even if the storage and reading of the charge information before the irradiation is only halfway through an image, according to the image of a divided area corresponding to the halfway area, and an irradiation control device for controlling to carry out the irradiation after the storage and reading of the charge information before the irradiation are stopped by the storage and readout stopping device.

3. The imaging apparatus according to claim 1, comprising a second readout setting device for reading the charge information before the irradiation periodically, and interposing, in an arbitrary cycle, a non-reading operation between the reading in that cycle and reading in a following cycle.

4. The imaging apparatus according to claim 3, comprising a readout stopping device for stopping the reading of the charge information before the irradiation, even if the reading of the charge information before the irradiation is only halfway through an image, according to the image of a divided area corresponding to the halfway area, and synchronously with a cycle corresponding to halfway timing, and an irradiation control device for controlling to carry out the irradiation after the reading of the charge information before the irradiation is stopped by the readout stopping device, and at a time of the non-reading operation.

5. The imaging apparatus according to claim 4, wherein the reading of the charge information before the irradiation is carried out periodically in order of divided adjoining areas, and when a last area is finished, a return is made to a first area for repetition.

6. The imaging apparatus according to claim 5, wherein the reading of the charge information at an irradiation time is started from a next area adjacent the area where the reading of the charge information is stopped, the reading of the charge information at the irradiation time is carried out periodically in the order of the divided adjoining areas from the starting area, and when the last area is finished, a return is made to the first area for repetition.

7. The imaging apparatus according to claim 5, comprising an area changing device capable of changing areas where the reading of carriers at the irradiation time is started, wherein the reading of the charge information at the irradiation time is carried out periodically in the order of the divided adjoining areas from the starting area, and when the last area is finished, a return is made to the first area for repetition.

8. The imaging apparatus according to claim 7, wherein the area changing device is arranged to start the reading of the charge information from the first area.

9. The imaging apparatus according to claim 5, wherein the reading of the charge information is carried out continuously according to all areas of the image.

10. The imaging apparatus according to claim 1, comprising a correcting device for correcting charge information read at an irradiation time based on charge information read at a non-irradiation time of the light or radiation.

11. The imaging apparatus according to claim 10, wherein the charge information read at the non-irradiation time for use in the correction is the charge information read before the irradiation.

12. The imaging apparatus according to claim 10, wherein the charge information read at the non-irradiation time for use in the correction is charge information read after the irradiation.

13. The imaging apparatus according to claim 10, wherein the charge information read at the non-irradiation time for use in the correction consists of more than one piece.
